(19) United States
(12) Patent Application Publication
(10) Pub. No.: US 2005/0175187 A1
(43) Pub. Date: Aug. 11, 2005

(54) Active noise control system in unrestricted space

(76) Inventors: Selwyn Edgar Wright, Huddersfield (GB); Branislav Vuksanovic, Oakwood (GB); Hidajat Atmoko, Huddersfield (GB)

Correspondence Address: HARRINGTON & SMITH, LLP, 4 RESEARCH DRIVE, SHELTON, CT, 06484-6212, US

(21) Appl. No.: 10/511,125

(22) PCT Filed: Apr. 14, 2003
(86) PCT No.: PCT/GB03/01565

(30) Foreign Application Priority Data
Apr. 12, 2002 (GB) 0208421.8

(51) Int. Cl. A61F 11/06; G10K 11/16; H03B 29/00

(52) U.S. Cl. 381/71.12

(57) ABSTRACT

The adaptive speed to changes in a primary source noise is increased through a noise detecting means feeding parallel multi-passband means and multi-transverse adaptive filter means, where each adaptive filter has its own individual adaptive step size means adjusted automatically according to the signal strength at each passband output. The output from each of the multi-adaptive filter means drives a secondary canceling source generating means where each multi-adaptive filter means is automatically adjusted to produce minimum sound in its passband at an error detecting means. Alternatively, the output from the noise detecting means is negated through a negation means, passed through a plant neutralization inverse means and an inverse delay means before driving the secondary source generating means. The secondary source output is aligned and match in amplitude to that of the primary sources, through a delay buffer means and an amplitude regulator means, which are adjusted successively until the output at the error detector means is a minimum.

### FIELD OF INVENTION

The present invention relates to a noise control system, which is preferably an active noise control system, and a method for controlling noise, particularly but not exclusively in large unrestricted spaces.

### BACKGROUND OF INVENTION

Conventional adaptive cancellation systems using traditional transverse finite impulse response (FIR) filters, together with least mean square (LMS) adaptive algorithms, well known in the prior art, are slow to adapt to primary source changes. This makes them inappropriate for cancelling rapidly changing noise, including unpredictable noise such as speech and music. Secondly, the cancelling structures require considerable computational processing effort to adapt to primary source and plant changes, particularly for multi-channel systems.

A general structure for such a cancellation system is shown in the applicant's international application having publication no. WO 01/63594. Here a primary source to be cancelled, a cancelling secondary source and an error sensor are in successive substantial alignment. Noise emanating from the primary source is cancelled using the second noise source and optimum cancellation is achieved by measuring the error between the unwanted primary noise and the actual noise produced by the second source. This error is fed to a system of FIR filters as a feedback for adjusting the noise produced by the second noise source.

These FIR filters adapt with increasing speed (reduced time constant) in reducing the noise, as the number of transverse control taps (coefficients) in the filter is increased to an optimum value. The adaptive speed at which the cancellation noise adapts to match the unwanted noise increases with the cancelling strength β=μA², where μ is the adaptive step size of the cancellation noise with each adaptive iteration and A is the peak signal amplitude of the cancellation noise. The speed also decreases with increases in the spectrum density. Thus for a primary source with frequencies of various amplitudes, the adaptive speed will reduce as the number of source frequencies increases, with the lower amplitudes adapting more slowly. If the signal is non-varying, then the lower amplitude frequencies will adapt eventually, given sufficient taps and time. But for source frequencies varying in time the smaller amplitudes will not have time to catch up (adapt completely), producing slow adaptation and signal distortion.

Further disadvantages of the conventional transverse FIR adaptive systems are (i) basic instability, where the error sensor is permanently required and functioning to maintain stability (ii) deteriorated cancellation away from the error sensor and (iii) susceptibility to environmental changes, through a large controlling propagation distance.

### SUMMARY OF THE INVENTION

According to a first aspect of the present invention there is provided a noise control system as set out in claim 1.

According to a second aspect of the present invention there is provided a noise control system as set out in claim 7.

Various preferred or optional features are defined in the other claims.

### BRIEF DESCRIPTION OF DRAWINGS

Embodiments of the invention will now be described, by way of example only, with reference to the accompanying drawings in which:

FIG. 1 is a block diagram of a multi-bandpass, variable μ, fixed β, transverse FIR adaptive filter, in accordance with a first embodiment;

FIG. 2 is a block diagram of an instantaneous plant inverse negative direct replica cancelling (IPINDR) system, in accordance with a second embodiment;

FIG. 3 is a diagram illustrating signal alignment in sample numbers; and

FIG. 4 is a block diagram of multi-channel configurations using the IPINDR approach of the second embodiment.

### DESCRIPTION OF PREFERRED EMBODIMENTS OF THE INVENTION

In the figures, like reference numerals indicate like parts and multiple like elements are denoted using lower case letters as sub-reference numerals.

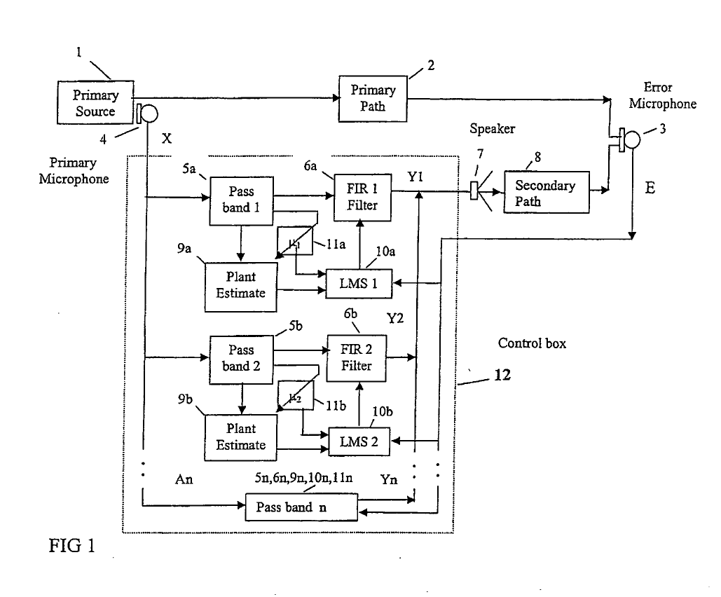

Referring firstly to FIG. 1, there is shown a multi-passband, variable μ, fixed β method to increase the adaptive speed of transverse FIR filters to primary source changes. The approach is to divide the source spectrum bandwidth into frequency pass-bands, where each passband has a separate FIR filter with its own μ made inversely proportional to A² in each passband, tending to maintain a constant β and therefore adaptive speed, irrespective of the spectrum amplitude. Thus a faster and similar response of the cancellation sound to the unwanted noise, will be more nearly obtained as the number of passbands increase for a given total spectrum bandwidth.

FIG. 1 shows a primary noise source 1 which produces a primary noise to be cancelled. This noise is shown to propagate along a primary path 2. There is further shown a primary transducer in the form of a microphone 4 disposed close to the primary noise source 1, and arranged to feed measured reference sound ‘x’ into a control box 12. An output from the control box 12 is arranged to be fed to a speaker 7, which produces a secondary cancelling sound that passes along a secondary path 8. Noise from both the primary and secondary paths is arranged to be received by an error transducer in the form of a microphone 3. The output error signal E of this microphone 3 is fed into the control box 12.

In operation, the noise from the primary source 1 propagates along the primary path 2 to be received at the error microphone 3. The noise is also measured in close proximity to the primary source 1, using the microphone 4. The resulting secondary signal x from the microphone 4 representative of the primary source noise is then fed into the control box 12.

Within the control box 12 there are provided a number n of pass band and filter arrangements. Only the first two of these is shown in detail, and a variable number of further arrangements can be added as required, as will be explained below. The first of these two arrangements comprises a passband 1, labelled with reference numeral 5a, a conventional finite impulse response (FIR) 1 filter 6a, a conventional control system transfer function estimate 9a including estimates of the elements 4,7,8 and the computational implementation (not shown), a conventional least mean square (LMS) or its equivalent algorithm 110a and an adaptive step size 11a.

The second passband and filter arrangement comprises corresponding elements labelled with the sub-reference numeral b. Similarly, each of the n arrangements has corresponding “in” elements.

The above-mentioned secondary signal from the microphone 4 is passed into each of the n passband filters 5n. The process will be described with reference to the first passband and filter arrangement. Thus the secondary signal is passed into passband 15a, and an output from this filter is passed through the FIR 1 filter 6a, to the secondary transducer, loud speaker 7. The loud speaker 7 generates the secondary cancelling sound that propagates through the secondary propagation space 8 to the error microphone 3, as mentioned previously.

The output from the passband 1 filter 5a is also passed through the control system estimate 9a and the output of the plant estimate 9a is then passed into the least mean squared LMS 1 algorithm 10a. Also fed into the LMS 1 algorithm 10a is the error signal E from the error microphone 3 and the adaptive step size 11a, which is automatically calculated from the passband 15a output level such that the adaptive step size is adjusted proportional to A² with each adaptive time step. The output from the LMS algorithm 10a is passed into the FIR filter 1 to control the FIR 1 filter 6a adaptive process so as to drive that part of the error signal E caused by the pass-band 1 to a minimum.

Similarly, the output from the primary microphone 4 is passed into the passband 2 filter 5b, through the FIR 2 filter 6b into the secondary loud speaker 7. The loud speaker 7 generates the secondary sound that propagates through the secondary propagation space 8 to the error microphone 3. The output from the pass-band 2 filter 5b is passed through the same control system estimate 9b, then into the LMS 2 algorithm 10b, together with the error signal E from the error microphone 3 and the output from the automatic adaptive step size 11b, whose size is determined by the output from passband 2 filter 5b. The output from the LMS algorithm 10b then controls the FIR 2 filter 6b adaptive process to drive the error signal in its passband to a minimum.

To extend the total frequency bandwidth or reduce the spectrum energy per passband, additional ‘n’ passband adaptive systems, each equalizing the adaptive speed in each of its passbands, can be added. The number of passbands will therefore depend on the spectrum density, the total spectrum bandwidth and the speed of adaptation to variations in the noise x required.

As the adaptive strength β and therefore speed, is proportional to the peak signal amplitude ‘A’ squared times the adaptive step size μ in each passband, then if the step size is reduced proportional to the signal amplitude squared, automatically, then the adaptive strength β will be maintained within the passband irrespective of amplitude.

Applying the same technique in each passband will tend to give an equal response to all frequencies in all the passbands. This increases the overall adaptive speed and reduces the spectrum distortion compared with a conventional transverse FIR filter. In other words, this embodiment increases the adaptive speed of the system to cancel the primary noise evenly across the frequency spectrum as the primary noise varies, thus reducing the signal distortion. However, the method has a maximum adaptive speed limited by a finite cancelling strength β. As β increases the stability bandwidth shrinks, its maximum value is given by the stability zero band width, as considered by Wright et al, Journal of Sound and Vibration (2001) 245(4).

The approach of the embodiment of FIG. 1 is therefore an improvement over prior art systems and is adequate for moderately changing primary sources such as unsteady periodic noise. It can have the disadvantage of intensive computation as it requires adaptive FIR filters and FIR passband filters for each band, although the passband filters could be implemented into hardware to reduce the computational burden.

To implement a really fast response to source changes, including unpredictable noise, and avoiding the disadvantages of the first embodiment, the online adaptive transverse FIR filters are removed and the primary source signal cancelled with a negative copy of itself, directly.

A time domain solution that gives virtually instantaneous response to primary source changes and is computationally efficient, is to negate a copy of the primary source signal, compensate for signal distortion caused through hardware implementation of the secondary cancelling system, align and match the resulting secondary wave with the primary wave at its instantaneity point.

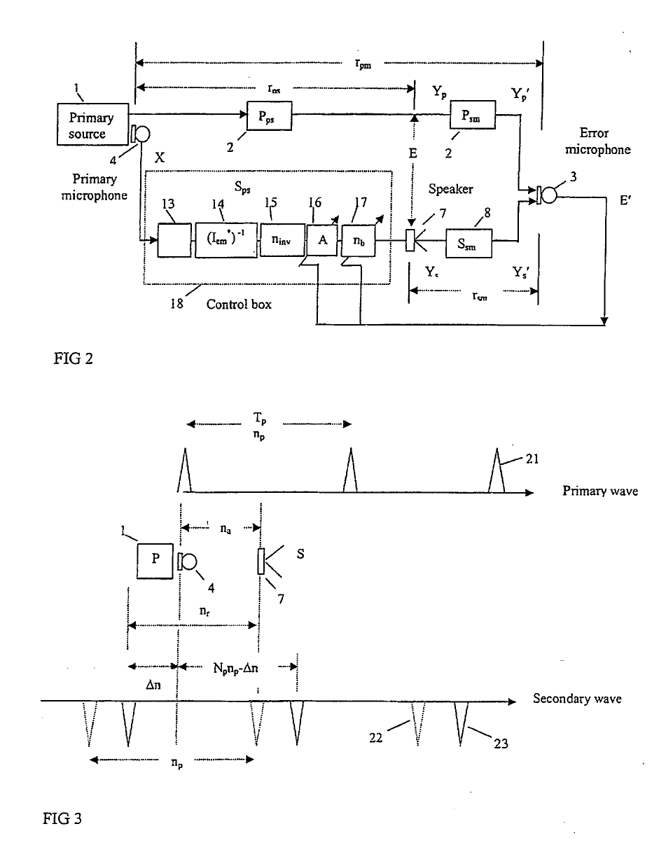

A second embodiment of the invention, as shown in FIG. 2 is arranged to achieve these advantages and mitigate the disadvantages of the first embodiment of FIG. 1.

To increase the response to rapidly changing primary sources, to avoid the disadvantages of conventional adaptive FIR filters discussed earlier, and to reduce the computational effort, the control box 12 in FIG. 1 is replaced with a control box 18 in FIG. 2. The process used to deal with the cancellation of arbitrary noise, including non-periodic unpredictable noise, is described generally in the time domain. Again to generate the secondary cancelling signal, a copy x of the primary source signal is measured using the primary microphone 4.

The control box 18 contains a negator 13, a control system neutralisation inverse estimate 14, an inverse delay required to obtain the inverse system estimate 15, an amplitude control 16 and an adjustable sample delay buffer 17, all arranged in series. The error signal E from the error microphone 3 is passed into each of the attenuation regulator 16 and the adjustable sample delay 17.

In operation, the output from the primary microphone 4 is negated in negator 13, and then convolved with the control system neutralization inverse estimate 14, which removes the signal distortion produced by the cancelling system hardware. The control system inverse 14, for example, in the form of an FIR filter can be measured directly in series with the control system. For non-minimum phase inverse functions the delay n^{Inv}, 15 is used in parallel with the control system and its inverse to realize these functions. This delay effectively becomes part of (series with) the inverse system estimate.

An alternative is to determine the system inverse from its impulse response measured in parallel with the control system. Then the inverse can be obtained through the frequency domain, as described below under the heading “Inverse Functions”.

The signal is then passed through the amplitude control 16 and the adjustable delay buffer 17, and then to the secondary loud speaker 7, where the resulting secondary signal Y propagates through the secondary propagation space 8, arriving at the error microphone 3 as Yₛ′. The signal from the primary source passes along the primary path 2 to the error microphone 3 as before, and is labelled in FIG. 2 as Yₚ′.

Details of operation and characteristics of the system of FIG. 2 will now be described.

Cancelling Characteristics

The instantaneous, plant inverse, negative direct replica (IPINDR) system has the following characteristics:
1) Secondary cancelling signal is ‘copied’ from the primary source using a primary sensing transducer (microphone or equivalent), suitably isolated from the secondary source (shielding and/or directional transducers) to prevent feedback between the two.
2) Secondary signal is negated in preparation for cancelling the primary signal.
3) The electromechanical system (impulse) response Iₑₘ, which produces distortion in the cancelling signal, is neutralised/reduced by (i) physically altering the dynamic response of the system, particularly the dominant component, namely the sound transducer (the loud speaker 7) together with its power amplifier (not shown), (ii) mathematically modifying the net response of the system through adding the appropriate poles/zeros to the overall transfer function, (iii) measuring the impulse response of the system and inverting. The system includes essential components in the secondary sound cancelling path (computer A/D, D/A converters, aliasing/quantisation filters, amplifiers microphones and loudspeakers)
4) The physically modified control system, and/or the neutralised control system is used to drive the secondary source (i.e. the cancelling loud speaker 7).
5) The resulting secondary acoustic wave is combined and aligned with the primary acoustic wave by appropriately positioning the secondary source 7 downstream of the primary source 1 in the direction of the wave propagation and the error microphone 3. This facilitates a time advance relative to and along the primary wave represented by the shift function h(t+τₐ), where τₐ is the time advance and is given by τₐ=rₚₛ/Cₒ.
6) The time advance τₐ is the wave propagation time between the primary 1 and secondary 7 sources, rₚₛ is the propagation distance between the sources and cₒ is the propagation speed (speed of sound). The time advance is necessary to offset the cancelling signal processing delay represented through h(t−τᵣ), where τᵣ is the secondary path processing time retardation.
7) The cancellation is dependent on the distance between the primary 1 and secondary 7 sources, rₚₛ=rₚₘ−rₛₘ (rₚₘ is the primary source microphone-error microphone distance), not on the secondary source-error microphone distance rₛₘ, as in the case of the conventional prior art adaptive FIR cancelling process. The controlling distance rₚₛ is considerably smaller than the controlling distance rₛₘ. This makes this critical propagation space much less vulnerable to environmental changes, such as fleeting reflections, than in the conventional adaptive FIR method.
8) Acoustically the primary and secondary sources form a phase controlled dipole (PCD), as described in Journal of Sound and Vibration (2001) 245 (4). Here the phase of the secondary source is adjusted to be out of phase with the primary sound field at the error microphone 3 located downstream in successive alignment following the primary 1 and secondary 7 sources. The resulting radiated acoustic field directivity (shadow shape) can be adjusted to be progressively tripole (cardiod), dipole (figure of eight) and quadrupole (four leaf clover), as the difference between the primary 1 and secondary 7 source distance rₚₛ increases.
9) The PCD, in this direct negative replica case, uses the propagation distance rₚₛ for both the primary and secondary waves. This produces exact alignment between the waves, giving maximum shadow at all points along the wave from the primary source, in the direction of the error microphone 3. By contrast, in the conventional adaptive FIR system, the propagation distance rₚₘ is used for the primary path and rₛₘ for the secondary path. This produces exact alignment only at the error microphone, giving a slight phase difference at all other points along the wave, progressively deteriorating the shadow with distance.
10) The IPINDR cancelling system of FIG. 2 is inherently stable requiring the error microphone 3 only to set up the cancellation process. After the setting up, the cancellation is self-sustaining, without the use of the microphone 3, except for all but severe environmental changes.
   Mathematical Description

Equivalent control concepts apply equally to analogue systems, but modern digital systems are more precise and do not suffer from drift. The control is therefore described in terms of digital control. For these systems, the control is implemented through samples generated by the sampling frequency fₙ. The time advance τₐ=rₚₛ/Cₒ, where cₒ is the speed of sound, is equivalent to a sample advance number of:
nₐ=τₐfₙ=rₚₛfₙ/cₒ (1)

The total sample delay (retardation) nᵣ is generated through (i) the unavoidable secondary control system implementation time delay nᵢₘₚ, including the control system inverse delay nᵢₙᵥ needed to retard advanced inverse functions (as calculated in the control system delay 15) and (ii) an adjustable sample delay n_{b} intentionally added through the delay buffer 17 (or equivalent means) to fine tune off line, or momentarily on line, signal alignment, particularly through considerable environmental changes.

This gives a total sample retardation number:
nᵣ=nᵢₘₚ+n_{b}, nᵢₘₚ≈nᵢₙᵥ (2)

For a periodic wave, the secondary wave alignment with the primary wave (as illustrated in FIG. 3 to be described in more detail below), is given by:
Nₚnₚ−Δn=0, Δn=nᵣ−nₐ, nₚ=Tₚ/Tₙ=fₙ/fₚ (3)

Where nₚ is the number of samples in the period Tₚ of the primary wave of periodic frequency fₚ and Nₚ is the period number that the primary wave is in advance of the secondary wave giving:
nₐ=nᵣ−Nₚnₚ (4)

For a slowly changing periodic noise the system can be non-causal i.e. the delay τᵣ can be longer than the advance τₐ, as here only the periods need to be aligned i.e. Nₚ can be any integer. For unpredictable noise the signals must be causal and exactly aligned, and the advance must balance the delay exactly, i.e. Nₚ=0, making
h(t+τₐ)h(t−τᵣ)=h(t+τₐ−τᵣ)=h(t) (5)

The sample advance nₐ is adjusted by adjusting the distance between the primary and secondary source rₚₛ, according to equation (1), until nₐ is approximately the same as but greater than nᵣ. The delay buffer n_{b} in equation (2) is then fine tuned until nₐ=nᵣ, giving a minimum error E at the error microphone 3. The amplitude A of the secondary signal is adjusted to match that of the primary source signal giving a minimum error E at the error microphone 3.

The last two steps are successively repeated, manually or automatically, until the lowest minimum error E is achieved. This indicates that the secondary and primary signals are in alignment at the error microphone 3, and at all points along the wave.

Correlation Process

Referring back to FIG. 2, the difference between the primary signal Yₚ′(t) and secondary signal Yₛ′(t) at the error microphone becomes:
E′(t)=Yₚ′(t)−Yₛ′(t)=X(t)*[Pₚₛ*Pₛₘ−Iₑₘ*)⁻¹, *SₚₛSₛₘ] (6)

Where * indicates linear convolution, x(t) is the reference signal at the primary source, Pₚₛ and Pₛₘ are the primary path responses, i.e. primary to secondary source and secondary source to microphone, respectively. Iₑₘ is the actual electro-mechanical control system impulse response of the cancelling system and (Iₑₘ*)⁻¹ is the measured or calculated inverse of the electromechanical control system impulse response. Sₚₛ and Sₛₘ are the primary-secondary source computation delay and secondary source-microphone path responses, respectively.

If the propagation path terms Pₛₘ=Sₛₘ, and Pₚₛ=h(t−τₐ) is a pure delay, and further the total computation delay Sₚₛ=A.h(t−τᵣ) where A is an amplitude adjustment, then the difference signal at the secondary loud speaker becomes:
E(t)=Yₚ(t)−Yₛ(t)=X(t)*Sₛₘ(t)*[h(t−τₐ)−A. Iₑₘ*(Iₑₘ*)^{-1*}h(t−τᵣ)] (7)

For a time varying periodic noise or unpredictable noise, the signals have to be matched exactly. Thus the zero order period Nₚ=0 has to be used giving nₐ=nᵣ and τₐ=τₐ.

In this case, equation (7) becomes in the frequency domain:
E(f)=Yₚ(f)−Yₛ(f)=X(f)Sₛₘ(f)[1-A.B(f)/B*(f)e^{j(θ-θ*)}] (8)

Where f is the acoustic frequency, h(f)=e^{j2πfτr}, B and B* are the amplitudes and θ and θ are the phases of the impulse response Iₑₘ and estimated (measured) response Iₑₘ* respectively. For zero frequency distortion, the plant dynamics has to be neutralised completely, from equation (8)
nₐ=nᵣ, A=B*/B, θ*=θ giving E=0 (9).

There is a minimum distance rₚₛ between the primary and secondary source for cancellation to be achieved. This is determined by the secondary path processing time which is basically the delay nᵢₙᵥ required in the inverse function realization. From equations (1), (2) and (9) this distance is given by
rₚₛ=nᵢₙᵥcₒ/fₙ (10)

This is the minimum distance for the cancellation of unpredictable noise to succeed. nᵢₙᵥ can be large for non minimum phase control system functions.

Thus the secondary signal Yₛ′ is aligned with the primary signal Yₚ′, initially by adjusting, approximately, the distance rₚₛ in equation (10), and then fine tuning by adjusting the sample delay buffer n_{b} 17 to give minimum error E at the error microphone 3. The amplitude of the secondary signal is matched to that of the primary signal by adjusting the amplitude at the amplitude adjustment 16, to give a minimum error at the error microphone 3. The amplitude A and the delay n_{b} are then successively adjusted until a minimum error is achieved at the error microphone 3, manually or automatically.

Referring again to FIG. 3, this figure illustrates the secondary signal alignment with the primary signal in sample numbers. The primary source 1 is shown to produce a primary wave 21 of period Tₚ propagating rightwards in the figure, where nₚ is the number of samples in the period Tₚ and Nₚ is the period number that the primary wave 21 is in advance of a secondary wave produced from the secondary source 7. The secondary wave position as measured from the primary microphone 4 and outputted directly from the loudspeaker 7, without any delay between the primary microphone 4 and the loudspeaker 7 is shown by the dashed representation 22.

Moving the speaker rightwards in the figure by nₐ samples also moves the secondary wave with it and advances its time compared to the primary wave 21. The position of the secondary wave after including a processing delay nᵣ is shown by the solid representation 23. For cancelling steady periodic noise the periods need only to be aligned (Nₚ integer in equation (4)). For unpredictable noise the secondary signal needs to be aligned exactly with the primary signal (Nₚ=0). This is accomplished by adjusting the propagation distance between the secondary loudspeaker 7 and the primary source nₐ to equal that of the computation delay nᵣ, making Δn=0 in equation (3).

Shadow Bending

Shadows are formed at an angle α_{B} from the line joining the primary 1 and secondary 7 sources, from equation (1)
n_{B}=Δrₚₛfₙ/cₒ, Δrₚₛ=rₚₛ−rₚₛ′=rₚₛ(1-cos α_{B}) (11)
where n_{B} is the buffer sample change, rₚₛ′ is the propagation distance in the direction of the shadow minimum. Rearranging the above equation gives
α_{B}=cos-1[(fₙrₚₛ−n_{B}cₒ)/fₙrₚₛ] (12)

The shadow bending or rotation from the source axis, per n_{B}, therefore depends on the relative magnitude fₙrₚₛ compared to cₒ.

Inverse Functions

To obtain minimum distortion of the cancelling process, resulting in maximum cancellation, it is important to implement neutralisation of the secondary control system response. This can be obtained through an accurate measurement of the inverse of the actual electromechanical control system impulse response (Iₑₘ)⁻¹.

An estimate of (Iₑₘ*)⁻¹ can be obtained in the time domain, directly in series with the actual Iₑₘ, off-line, using a white noise training signal. Care is needed in performing direct inverse estimates, as inverted functions are potentially unstable. For example, proper functions (functions with more poles than zeros) become improper functions when inverted. More seriously, ‘unstable’ zeros lying outside the unit circle in the Z domain (non-minimum phase functions) become unstable poles, turning delays into advances, when inverted. For these advanced functions in negative time to be to realized (i.e. for the adaptive process to converge effectively), a delay nᵢₙᵥ is required in parallel with the training process to delay these functions into real (positive) time.

A method that does not require a training delay is to obtain the inverse directly from the impulse response. An estimate Iₑₘ* is measured in parallel with the actual Iₑₘ, using a white noise training signal. The spectrum amplitude B and phase θ are then obtained through performing the discrete fast Fourier transform (FFT) or swept spectrum or equivalent on Iₑₘ* thus:
FFT(Iₑₘ*)=Σ(Be^{jωθ}) (13)

The inverse is then obtained by simply inverting B and negating θ, and then reassembling them back into the time domain, thus the inverse fast Fourier transform (IFFT) becomes:
IFFTΣ(Be^{jωθ})=Σ(B⁻¹e^{-jωθ})=(Iₑₘ*)⁻¹ (14)

A delay to retard the function can be added later-as required.

Multi-Channel Systems

A single channel PCD cancelling system produces a narrow cancellation region (shadow). For practical systems requiring wide shadows, particularly at high frequencies, multi-channel (multi-secondary source-multi error detector) systems are required, to generate a practical shadow over a wide well defined angle. The primary source microphones, secondary cancelling sources and error microphones are generally arranged in successive planes or arcs from the primary source and contained within defining control angles, forming boundaries for the acoustic shadows, as described in International publication no. WO 01/63594.

For these multi-channel systems to operate effectively, the sound propagation path differences (Δr_{pd}) between the various combinations of cancelling speakers and error microphones of multiples (p) of acoustic half wavelengths (λₚ=cₒ/fₚ where fₚ is a series of frequency peaks) should be avoided, which is also described in International Publication no. WO 01/63594, giving
Δr_{pd}=pcₒ2fₚ where p=1,2,3, . . . etc. (15)

Or in terms of sample numbers, for a discrete sampling system, n_{pd}=t_{pd} fₙ where t_{pd}=Δr_{pd}/cₒ, the uncontrollable sample numbers and uncontrollable frequencies to be avoided are
n_{pd}=pfₙ/2fₚ or fₚ=pfₙ/2n_{pd} (16)

Generally, IPINDR multi-channel systems are fundamentally stable i.e. they do not require the error microphone to maintain cancelling stability. The cancelling system is basically instantaneous to the response of primary source changes, as a negative copy of the primary source signal is passed directly through the secondary source system to the cancelling loud speaker. Apart from the convolution, there are no computational demanding processes either. A simple phase and amplitude error adjustment is effected using a simple delay buffer and amplitude regulator.

Therefore, for a non-changing control system, the error microphone can be dispensed with after the initial setting up to produce minimum error (sound). Each channel can be set up independently, requiring no inter-channel coordination. Of course a multi-channel computer coordinated system should always out-perform a set of independent channels.

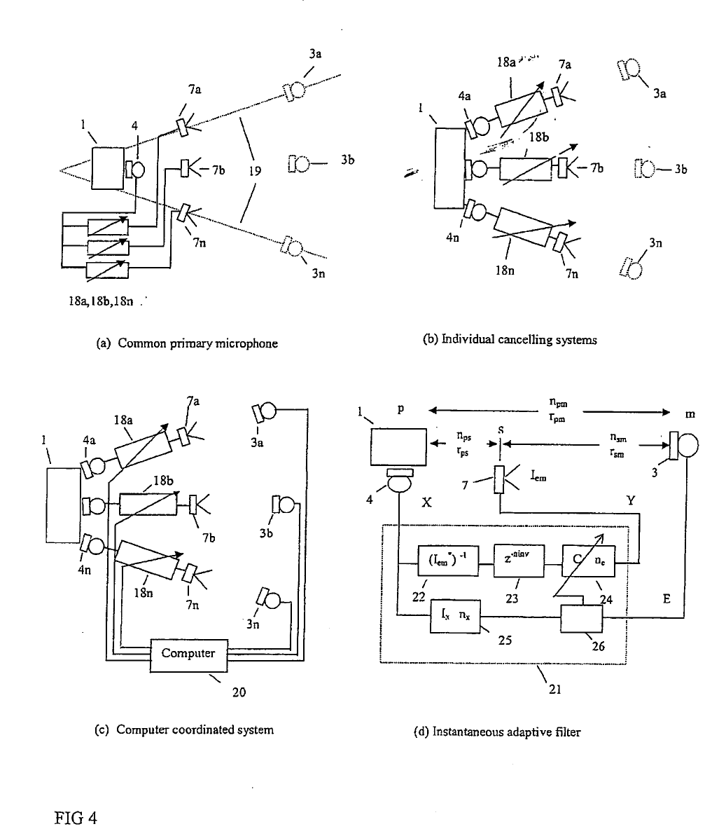

FIG. 4 shows four possible configurations. Although these configurations are shown with respect to the second embodiment (IPINDR system), they could be used with respect to the first embodiment (of FIG. 1) with the exception that each channel requires a permanent error microphone. In this case, where the control boxes 18 and 21 are shown, control box 12 would be substituted.

FIG. 4(a) shows the configuration for a small or large in-phase primary source 1 generating a shadow over an angle 19. Here a single primary microphone 4 is sufficient to drive all the secondary sources 7. A single error microphone 3 is sufficient to adjust each channel, one at a time, at each of the angle positions, as indicated with the dotted outline. Within the adjustable control boxes 18 are the adjustment control elements including the amplitude regulator A and the delay buffer n_{b} shown in the chain dotted box 18 in FIG. 2. The secondary sources 7 and error detectors 3 are arranged generally in successive planes or arcs from the primary source and contained within control angles 19 forming shadow angles, both horizontally and vertically (not shown).

FIG. 4(b) is a configuration for an out of phase primary source I (for example modal distributions within a metal structure). Here separate primary microphones 4 are used to measure the local sound variations across the primary source and drive each channel separately, making them self-contained units. Each unit consists of a primary microphone 4, control system 18, and loud speaker 7. Again only a single error microphone is used in turn, at each angular position, to minimise the error signal for each channel, one at a time and then as a group.

The amplitude A and delay n_{b} adjustments in control box 18 can be coordinated through computer control to align channels to give a collective minimum error at the error sensors for off-line adjustment, or momentary on-line adjustment for severe environmental changes. These control elements can also be replaced with, for example, a simple C filter (few taps FIR transverse filter and a modified filtered x algorithm), as in the control box 21 (see below).

FIG. 4(c) shows such a computer coordinated multi-channel system. An array of units 4, 18 and 7 and an array of permanent error microphones 3 are shown in full line. Each of the error microphones 3 and control boxes 18 is linked to a computer 20. The control elements, amplitude A and delay n_{b}, in control box 18, are adjusted automatically through the computer 20 to produce a minimum collective error at the error microphones 3.

Sound propagation path differences between the secondary sources 7a,7b,7n and the error detectors 3a,3b,3n of multiples of acoustic half wavelengths should be avoided for these multi-channel systems to operate effectively, as described in equations (15) and (16) above. All configurations are capable of shadow angle rotation through appropriate adjustment of n_{B}, rₚₛ or fₙ.

FIG. 4(d) shows the details of a further example of a computer-adjusted system. The control box 18 is replaced with the control box 21. Element 22 is the measured control system inverse, element 23 is the inverse delay required to obtain the inverse, element 24 is a fine adjustment C filter (low order FIR transverse filter) and element 25 is the impulse response of the secondary path rₛₘ and control elements 22, 23 and 7.

The impulse response filters the reference signal x, from the primary microphone 4, before it is used in the adaptive algorithm 26 to align the primary and secondary waves. The adaptive algorithm 26 also uses the output from the error microphone 3 nₚₛ, nₛₘ, and nₚₘ are propagation distances in sample numbers between the primary source—secondary source 7, the secondary source 7—error microphone 3, and the primary source 1—error microphone 3, respectively. The relationships between propagating distances in samples and the secondary control system impulse response Iₛₘ, where z is the z domain discrete time transform, are:
nₚₛ+nₛₘ=nₚₘ and Iₛₘ=Iₑₘz^{-nsm}, (17)
giving the filtered x impulse response Iₓ as
Iₓ=(Iₑₘ*)⁻¹z^{-ninv}Iₛₘ=(Iₑₘ*)⁻¹Iₑₘz^{-nsm+ninv)} (18)
If Iₑₘ*=Iₑₘ
Iₓ=z^{-(nx)}, nₓ=nₛₘ+nᵢₙᵥ, and n_{c}=nₚₘ−nₓ=nₚₘ−nₛₘ−nᵢₙᵥ=nₚₛ−nᵢₙᵥ (19)

This adjustment scheme is practically instantaneous. If the control system inverse estimate is accurate i.e. Iₑₘ*=Iₑₘ, then the filtered x delay nₓ becomes simply the sum of the secondary path delay nₛₘ and the inverse delay nᵢₙᵥ and the C filter delay n_{c} becomes the difference between the primary secondary source delay nₚₛ and the inverse delay nᵢₙᵥ. For unpredictable noise n_{c}>0 making nₚₛ>nᵢₙᵥ. For predictable noise the C filter can also be used to reduce the minimum distance rₚₛ in equation (10). As the number of taps W_{c} decreases, the adaptive step size μ_{c} increases.

The applicant draws attention to the fact that the present invention may include any feature or combination of features disclosed herein either implicitly or explicitly or any generalisation thereof, without limitation to the scope of any of the present claims.

1. A noise control system comprising:
a primary sensor means arranged to detect a primary sound from a primary source and provide a first output signal indicative of the primary sound, which output signal has a frequency spectrum;
a sound producing means arranged to produce a secondary sound usable to cancel the primary sound;
an error sensor means arranged to sense a difference between the primary and secondary sounds and provide a second output signal indicative of the said difference;
a signal processing means arranged to receive and process the first and second outputs to produce a third output to be received by the sound producing means and used to produce the said secondary sound,
wherein the spectrum output from said primary sensor means is arranged to be divided into a plurality of frequency pass-bands, each frequency pass-band being arranged to feed a finite impulse response filter using an adaptive algorithm acting on the second output signal, and wherein each finite impulse response filter is arranged to produce an output signal, which output signals are combinable to produce the said third output signal.

2. A noise control system according to claim 1, wherein the adaptive algorithm acting on the second output signal is a least mean squared algorithm or equivalent.

3. A noise control system according to claim 1, wherein the sound producing means is arranged to adapt the secondary sound to the primary sound in a step-wise fashion having an adaptive step size adjusted proportional to the square of the amplitude within each pass-band.

4. A noise control system according to claim 1, wherein the sound producing means is arranged to modify the secondary sound until the second output signal is substantially minimised within each pass-band, thus tending to maximise the speed of modification of the secondary sound evenly across the frequency spectrum of the first output signal.

5. A noise control system according to claim 4, wherein the speed of modification of the secondary sound is substantially constant with amplitude of the primary sound.

6. A noise control system according to claim 1, wherein the adaptive algorithm comprises a control system estimate.

7. A noise control system comprising:
a primary sensor means arranged to detect a primary sound from a primary source and provide an output signal indicative of the primary sound; a sound producing means arranged to produce a secondary sound usable to cancel the primary sound;
a signal processing means arranged to receive the said output signal, calculate a negative substantial copy of the output signal and use the calculated negative substantial copy to produce an adjustment signal to be received by the sound producing means and used to produce the secondary sound.

8. A noise control system according to claim 7, wherein production of the adjustment signal compensates for control system distortion.

9. A noise control system according to claim 8, wherein the compensation is carried out by any or all of: (i) physically altering the dynamic response of the sound producing means, (ii) mathematically modifying the net response of the system through adding the appropriate poles/zeros to the overall transfer function and (iii) passing the signal through the control system inverse.

10. A noise control system according to claims 8, wherein a control system inverse is used to compensate for control system distortion and is obtained in series with the control system using a training signal derived from white noise or a periodic pulse train.

11. A noise control system according to claim 10, wherein the control system is a non-minimum phase function and a convergence delay is used in parallel with the control system and a training FIR filter to cause convergence of the training process.

12. A noise control system according to claims 8, wherein in order to compensate for control system distortion, a spectrum amplitude of a control system impulse response is obtained and inverted, a phase of the control system impulse response is obtained and phase negated and the inverted amplitude and negated phase are reassembled as a time domain inverse function.

13. A noise control system according to claim 12, wherein the inversion and negation is carried out using a Fast Fourier Transform and the reassembly is carried out using an inverse Fast Fourier Transform.

14. A noise control system according to claims 7, further comprising an error sensor means arranged to sense a difference between the primary and secondary sounds and provide an error signal indicative of the said difference.

15. A noise control system according to claim 14, wherein the signal processing means is arranged to receive the error signal for use in production of the adjustment signal.

16. A noise control system according to claims 7, wherein the sound producing means is disposed at a predetermined distance relative to the primary source such that sound takes a predetermined time to travel between the primary source and the sound producing means, and the signal processing means is arranged to use the predetermined distance to calculate the adjustment signal.

17. A noise control system according to claim 16, wherein there is a secondary signal processing sample delay associated with the sound producing means, which is useable to set a minimum predetermined distance and, which is offset by a signal advance, produced by positioning the sound producing means downstream of the primary source until the secondary sound is slightly in advance of the primary sound.

18. A noise control system according to claim 11, wherein there is a secondary signal processing sample delay associated with the sound producing means, which is useable to set a minimum predetermined distance and, which is offset by a signal advance, produced by positioning the sound producing means downstream of the primary source until the secondary sound is slightly in advance of the primary sound; wherein a dominant component of the signal processing sample delay is the said convergence delay.

19. A noise control system according to claims 7, wherein the secondary sound is substantially aligned and matched in amplitude with the primary sound by means of an adjustable circular buffer sample delay number and an amplitude adjuster, through successive adjustment.

20. A noise control system according to claims 7, wherein the primary sound and the secondary sound form a phase controlled dipole, where the combined sound directivity is controlled by adjustment of the distance between the primary source and the sound producing means.

21. A noise control system according to claim 20, wherein the propagation distances of the primary and secondary sounds are substantially identical such that the secondary sound is substantially completely aligned at all points along the primary sound, and thus produces a uniform shadow along the primary sound.

22. A noise control system according to claims 7, which produces acoustic shadows that are rotatable from a line joining the primary source and the sound producing means, the rotation angle depending on one or more of: a secondary signal processing sample delay change; a primary sound sampling frequency; a distance between the primary source and the sound producing means; and the speed of sound.

23. A noise control system according to claims 7, further comprising one or more further primary sensor means and one or more further sound producing means, arranged generally in successive alignment planes or arcs from the primary source and contained within shadow control angles.

24. A noise control system according to claim 23, wherein the shadow control angles extend vertically and horizontally.

25. A noise control system according to claim 23. further comprising one or more error sensor means arranged in the said successive alignment planes or arcs.

26. A noise control system according to claim 25 wherein the geometry of the system is adjusted by varying one or more of: the number of sound producing means; the number of error sensing means; distances between the sound producing means and the error sensing means; and the shadow control angles, so as to avoid the path differences between the sound producing means and the error sensing means over the operating frequency range being one or a multiple of the acoustic half wavelength of the sound producing means.

27. A noise control system according to claim 25, wherein each primary sensor means together with a respective sound producing means and error sensing means forms an independent channel, and each sound producing means is individually adjusted to minimise error at its respective error sensing means.

28. A noise control system according to claims 25, wherein the multiple primary sensor means, sound producing means and error sensing means form a group of channels, and the multiple sound producing means are collectively adjusted, under the control of a computing means, to produce a total collective minimum error at the error sensing means.

29. A noise control system according to claim 28, wherein the computing means uses an adjustment filter and a modified filtered algorithm of the output signal indicative of the primary sound, either off-line or momentarily on-line to align the channels and counteract control system changes, thus facilitating a substantially instantaneous adjustment of the secondary sound with respect to the primary sound.

30. A noise control system according to claim 29, wherein the C filter is further used to reduce the minimum distance of the sound producing means from the primary source at which the cancellation of predictable primary sound is achieved.

31. A method of controlling a noise, comprising the steps of:
detecting the noise and providing a first output signal indicative of the noise, which output signal has a frequency spectrum;
producing a secondary sound usable to cancel the primary noise;
sensing a difference between the primary noise and the secondary sound and providing a second output signal indicative of the said difference;
processing the first and second outputs to produce a third output to be used in the step of producing the said secondary sound,
dividing the spectrum output into a plurality of frequency pass-bands, and using each frequency pass-band to feed a finite impulse response filter and thus produce an output signal; and
combining the output signals to produce the said third output signal.

32. A method of controlling a noise, comprising the steps of:
detecting the noise and providing an output signal indicative of the noise;
producing a secondary sound usable to cancel the primary noise;
calculating a negative substantial copy of the said output signal and using the calculated negative substantial copy to produce an adjustment signal ; and
using the adjustment signal to produce the secondary sound.
